(19) United States
(12) Patent Application Publication
(10) Pub. No.: US 2019/0282652 A1
(43) Pub. Date: Sep. 19, 2019

(54) ABSORPTION PROMOTER FOR GARLIC COMPONENTS

(71) Applicant: KENKOUKAZOKU CO., LTD., Kagoshima-shi, Kagoshima (JP)

(72) Inventor: Hiromi TO, Kagoshima-shi (JP)

(73) Assignee: KENKOUKAZOKU CO., LTD., Kagoshima-shi, Kagoshima (JP)

(21) Appl. No.: 16/308,333

(22) PCT Filed: Jun. 5, 2017
(86) PCT No.: PCT/JP2017/020799

§ 371 Date: Dec. 7, 2018

(30) Foreign Application Priority Data
Jun. 10, 2016 (JP) 2016-115985

(51) Int. Cl. A61K 36/8962; A61K 35/57

(52) U.S. Cl. CPC A61K 36/8962; A61K 35/57

(57) ABSTRACT

The present invention relates to an absorption promoter for S-allyl cysteine (SAC) and alliin containing Garlic-Egg yolk.

### TECHNICAL FIELD

The present invention is related to an absorption promoter for garlic components.

### BACKGROUND ART

Garlic-Egg yolk is an excellent Japanese traditional food that has been popular in many households in southern Kyushu since the Edo period. “Traditional Garlic-Egg yolk” is prepared by mashing raw garlic, kneading this with egg yolk at low temperature and adding rice bran oil and the like to a Garlic-Egg yolk powder containing about 80% of garlic and about 20% of egg yolk.

Various active ingredients of garlic have been isolated along with the development of analytical science and technology and each component and its function has gradually been revealed. It is known that garlic contains sulfur-containing amino acids such as alliin, methinin, and cycloalliin, and peptides called such as γ-glutamyl-S-allyl-cysteine and γ-glutamyl-S-1-propenyl-cysteine are included. Moreover, a preventive effect and growth inhibitory effect of garlic on prostate cancer and bladder cancer is disclosed in Patent Document 1.

Nonetheless, the characteristic function of Garlic-Egg yolk powder has not been fully revealed yet.

### PRIOR ART DOCUMENTS

### Patent Documents

Patent Document 1: JP 2001-302531 A

### SUMMARY OF INVENTION

### Technical Problem

The present invention provides an absorption promoter for S-allyl cysteine (SAC) and alliin.

### Solution to Problem

The present inventors have carried out dedicated research, and surprisingly found that the Garlic-Egg yolk powder has a superior absorption of S-allyl cysteine (SAC) and alliin as compared to garlic powder, and thus, completed the present invention. That is, the present invention is as shown below.

(1) an absorption promoter for S-allyl cysteine (SAC) and alliin comprising Garlic-Egg yolk;

(2) an absorption promoter for S-allyl cysteine (SAC) and alliin according to (1), wherein the Garlic-Egg yolk is a Garlic-Egg yolk powder.

### Advantageous Effects of the Invention

The present invention can promote the absorption of S-allyl cysteine (SAC) and alliin.

### DESCRIPTION OF EMBODIMENTS

In the present invention, Garlic-Egg yolk is a food formulated with Garlic-Egg yolk, and it can be utilized in the form of soft capsules, tablets, hard capsules, powder, and granules.

In the present invention, the Garlic-Egg yolk powder refers to a powdered Garlic-Egg yolk. Powdered Garlic-Egg yolk may be prepared as follows: Garlic is peeled, washed, steamed or heated under reduced pressure and kneaded, and when this is mushy, egg yolk is added and kneaded, which is then heated and dried to be in a powder state. Alternatively, it can also be prepared by processing each garlic and egg yolk in a powder form and mixing them.

Alliin is a natural sulfur compound contained in garlic. By cutting and grating, alliin turns into allicin causing odor resulting from the action of an enzyme in garlic called alliinase. When alliin is added to blood cells in vitro, increased phagocytic ability of leukocytes is observed.

S-allyl cysteine (SAC) is an odorless water soluble sulfur-containing amino acid that is generated by heat processing garlic, and a (colorectal) cancer prevention action and the like has been reported.

When subjected to heat processing, depending on the degree of treatment (temperature and period), flesh color changes from white to amber to black. The content of S-allyl cysteine is the highest when the color is amber, and when heated until the color turns to black, it decreases. On the other hand, it is less pungent and easier to eat than when the color is amber.

Garlic-Egg yolk of the present invention may be used for promoting the absorption of S-allyl cysteine (SAC) and alliin.

Also, Garlic-Egg yolk of the present invention can be used in soft capsules, tablets, hard capsules, powders, and granules.

Hereinafter, the present invention will be further described in details with reference to the Examples, but the present invention is not limited thereto.

### Example 1

A single dose of Test Food 1 (garlic powder) and Test Food 2 (Garlic-Egg yolk powder) was administered in a crossover study to 14 men of age between 20 and 39 years old and blood SAC and alliin concentration was measured over time and absorption was compared and examined.

TABLE 1

| | | |
|---|---|---|
| Allocated | Number of | Intake of Test Food |

| Group | Members | Phase I | Phase II |
|---|---|---|---|
| A | 7 persons | Test Food 1 | Test Food 2 |
| B | 7 persons | Test Food 2 | Test Food 1 |

Preparation Methods of Garlic Powder

The garlic was heated to 80 to 100° C. under reduced pressure while kneading and stirring was continued until it turned into a powder form. Then, dextrin was added for preparation so that the concentration of SAC and alliin would be equivalent to that of the Garlic-Egg yolk powder.

Preparation Methods of Garlic-Egg Yolk Powder

70 to 90 parts by weight of garlic was heated to 80 to 100° C. under reduced pressure while kneading, and after kneading this was returned to normal pressure. Next, 10 to 30 parts by weight of egg yolk was added at a temperature of 65° C. or less, and while kneading, the pressure was reduced, and then, the temperature was increased to 80 to 100° C. and mixed until it turned into a powder form, and thereby Garlic-Egg yolk powder was obtained.

Measurement Methods of SAC

High performance liquid chromatogram analysis (Column: 4.6×150 mm, Inertsil ODS-45 μm)

Mobile phase: phosphate buffer pH 2.6/methanol=85/15

### Detector: UV205 nm

Measurement Methods of Alliin

High performance liquid chromatogram analysis

### (Column: 4.6×150 mm, Inertsil ODS-45 μm)

Mobile phase: phosphate buffer pH 2.6/methanol=85/15

### Detector: UV205 nm

Composition of the test food per pack (per 3.5 g)

TABLE 2

| | Test Food 1 | Test Food 2 |
|---|---|---|
| | (Garlic powder) | (Garlic-Egg yolk powder) |
| Ingredients | Garlic powder, Dextrin | Garlic powder, Egg yolk powder |
| Analyzing | SAC 5 mg, | SAC 5 mg, |
| components | Alliin 33 mg | Alliin 33 mg |

| | |
|---|---|
| Preservation | Keep away from direct sunlight, Store at room temperature |
| methods | |
| Provider | KENKOUKAZOKU (Healthy Family) Co., Ltd. |

A pack of this (3.5 g) was wrapped in a thin edible layer of starch (oblaat) and a single dose was administered to a subject having an empty stomach with 180 mL of water.

Measurement of SAC and alliin was carried out before ingestion (0 h) and after ingestion (1 h, 2 h, 3 h, 4 h, 5 h, 6 h, 7 h, 8 h).

For AUC_{0-8H} of SAC and AUC_{0-8H} of alliin, the order effect and period effect were analyzed, and after confirming that the crossover design was appropriate, the comparison of Test Food 1 and Test Food 2 by crossover method was assessed using two-sample t-test. The calculation of AUC was made by the area calculated by trapezoidal rule with the value before the intake as a reference. In addition, it was also analyzed for the area above 0 (ΔAUC₀₋₈ₕ) after intake.

For Cmax, the comparison of Test Food 1 and Test Food 2 was assessed using one-sample t-test. As a reference, the concentration at each time point was evaluated in the same manner.

The numerical values were shown as mean±standard deviation and the significance level of the test was determined to be 5% at both sides.

SAC Concentration AUC₀₋₈ₕ

With respect to SAC concentration AUC₀₋₈ₕ, the order effect and period effect were examined and the order effect and period effect were significant and the crossover design was not appropriate.

TABLE 3

| | p value | | |
|---|---|---|---|
| Item | Food effect | Order effect | Period effect |
| Blood SAC concentration | 0.3681 | 0.0301 | 0.0162 |
| AUC_{0-8 h} | | | |

With respect to SAC concentration ΔAUC₀₋₈ₕ determined from variation, the period effect was significant; however, the order effect was not significant, and the crossover design was appropriate.

TABLE 4

| | p value | | |
|---|---|---|---|
| Item | Food effect | Order effect | Period effect |
| Variation of Blood SAC | 0.0234 | 0.2792 | 0.0001 |
| concentration AUC_{0-8 h} | | | |

SAC concentration ΔAUC0-8h was 3345.53±654.11 ng/mL·h in Test Food 1, and 3597.93±584.33 ng/mL·h in Test Food 2, and as for the food effect, Test Food 2 was significantly higher than Test Food 1.

TABLE 5

| | ΔAUC_{0-8 h} | ΔCmax | Tmax |
|---|---|---|---|
| Test Food | (ng/mL · h) | (ng/mL) | (h) |
| Test Food 1 | 3345.53 ± 654.11 | 524.73 ± 116.93 | 2 |
| (Garlic powder) | | | |
| Test Food 2 | 3597.93 ± 584.33^{##} | 562.45 ± 141.89 | 2 |
| (Garlic-Egg yolk | | | |
| powder) | | | |

Alliin Concentration AUC₀₋₈ₕ

With respect to alliin concentration AUC_{0-8H}, order effect and period effect of crossover were examined. Here, it was found that period effect was significant: however, order effect was not significant, and crossover design was appropriate.

TABLE 6

| | p value | | |
|---|---|---|---|
| Item | Food effect | Order effect | Period effect |
| Blood alliin | 0.0031 | 0.5758 | 0.0000 |
| concentration AUC_{0-8 h} | | | |

Alliin concentration AUC_{0-8H} was 3253.74±701.79 ng/mL·h in the intake of Test Food 1 and 3613.51±588.18 ng/mL·h in the intake of Test Food 2, and as for the food effect, Test Food 2 was significantly higher than Test Food 1.

TABLE 7

| | ΔAUC_{0-8 h} | Cmax | Tmax |
|---|---|---|---|
| Test Food | (ng/mL · h) | (ng/mL) | (h) |
| Test Food 1 | 3253.74 ± 701.79 | 799.45 ± 266.72 | 1 |
| (Garlic powder) | | | |
| Test Food 2 | 3613.51 ± 588.18^{##} | 910.14 ± 217.74 | 1 |
| (Garlic-Egg yolk | | | |
| powder) | | | |

With respect to Alliin concentration ΔAUC₀₋₈ₕ also, period effect was significant; however, order effect was not significant, and crossover design was appropriate.

TABLE 8

| | p value | | |
|---|---|---|---|
| Item | Food effect | Order effect | Period effect |
| Variation of blood alliin | 0.0059 | 0.2758 | 0.0002 |
| concentration ΔAUC_{0-8 h} | | | |

Alliin concentration ΔAUC_{0-8H} was 3011.51±716.76 ng/mL*h in the intake of Test Food 1 and 3313.20±543.41 ng/mL*h in the intake of Test Food 2, and as for the food effect, Test Food 2 was significantly higher than Test Food 1.

TABLE 9

| | ΔAUC_{0-8 h} | ΔCmax | Tmax |
|---|---|---|---|
| Test Food | (ng/mL · h) | (ng/mL) | (h) |
| Test Food 1 | 3011.51 ± 716.76 | 769.18 ± 262.01 | 1 |
| (Garlic powder) | | | |
| Test Food 2 | 3313.20 ± 543.41^{##} | 872.60 ± 214.48 | 1 |
| (Garlic-Egg yolk | | | |
| powder) | | | |

SAC Concentration Cmax

According to the measured value, crossover design was inappropriate, and based only on the results of phase I, Cmax of Test Food 1 was 611.33±197.41 ng/mL and Cmax of the Test Food 2 was 503.04±126.11 ng/mL. The intake of Test Food 1 showed a higher value; however, no significant difference was recognized (two-sample t-test). Tmax at this time was 2 hours after the intake in both Test Foods 1 and 2.

With respect to the variation, Cmax of Test Food 1 was 524.73±116.93 ng/mL and Cmax of Test Food 2 was 562.45±141.89 ng/mL. Test food 2 showed a higher value; however, no significant difference was recognized (one-sample t-test). Tmax at this time was 2 hours after the intake in both Test Foods 1 and 2.

Alliin Concentration Cmax

According to the measured value, Cmax of Test Food 1 was 799.45±266.72 ng/mL and Cmax of Test Food 2 was 910.14±217.74 ng/mL, and Test Food 2 showed a higher value; however, no significant difference was recognized (one-sample t-test). Tmax at that time was an hour after the intake in both Test Foods 1 and 2.

For the variation, Cmax of the Test Food 1 was 769.18±262.01 ng/mL and Cmax of Test Food 2 was 872.60±214.48 ng/mL, and Test Food 2 showed a higher value; however, no significant difference was recognized (one-sample t-test). Tmax at that time was an hour after the intake in both Test Foods 1 and 2.

Conclusion of Efficiency

With respect to SAC concentration AUC_{0-8H} which is the major assessment item, according to the measured value, the crossover design was not appropriate; however, SAC concentration AUC_{0-8H} based on variation, the crossover design was appropriate. Test food 2 had a significantly higher ΔAUC_{0-8H} than that of Test Food 1.

Alliin concentration AUC_{0-8H} and alliin concentration ΔAUC_{0-8H} was appropriate for crossover design, and Test Food 2 had a significantly higher AUC_{0-8H} and ΔAUC_{0-8H} than that of Test Food 1.

With respect to SAC concentration Cmax and alliin concentration Cmax, no significant difference was recognized between Test Food 1 and Test Food 2.

Based on the above results, Test Food 2 (Garlic-Egg yolk powder) was confirmed to have a better absorption of SAC and alliin as compared to Test Food 1 (Garlic powder).

With respect to safety, no adverse events occurred, and problems were not recognized.

### INDUSTRIAL APPLICABILITY

The present invention can promote the absorption of S-allyl cysteine (SAC) and alliin.

1. An absorption promoter for S-allyl cysteine (SAC) and alliin comprising Garlic-Egg yolk.

2. An absorption promoter for S-allyl cysteine (SAC) and alliin according to claim 1, wherein the Garlic-Egg yolk is a Garlic-Egg yolk powder.

3. A method of promoting an absorption of S-allyl cysteine (SAC) and alliin comprising a step of administering Garlic-Egg yolk to a subject in need thereof.

4. The method according to claim 3, wherein the Garlic-Egg yolk is a Garlic-Egg yolk powder.
